(19) United States
(12) Patent Application Publication
(10) Pub. No.: US 2008/0029084 A1
(43) Pub. Date: Feb. 7, 2008

(54) DEVICE FOR IMPROVED PEPTIDE DELIVERY

(75) Inventors: Henry R. Costantino, Woodinville, WA (US); Roger C. Adami, Snohomish, WA (US); Connie Sau-Kuen Kwok, Bothell, WA (US)

Correspondence Address: NASTECH PHARMACEUTICAL COMPANY INC, 3830 MONTE VILLA PARKWAY, BOTHELL, WA, 98021-7266, US

(73) Assignee: NASTECH PHARMACEUTICAL COMPANY INC., Bothell, WA (US)

(21) Appl. No.: 11/774,492

(22) Filed: Jul. 6, 2007

Related U.S. Application Data
(60) Provisional application No. 60/821,528, filed on Aug. 4, 2006.

(51) Int. Cl. A61M 11/00; A61K 38/00; A61K 9/14

(52) U.S. Cl. 128/200.14; 424/489; 514/2

(57) ABSTRACT

What is described is a means for creating bimodal particle size distribution that targets both nasal cavity and pulmonary regions for drug delivery.

### BACKGROUND

This application claims the benefit under 35 U.S.C. §119(e) of U.S. Provisional Application No. 60/821,528 filed Aug. 4, 2006, which is incorporated herein by reference in its entirety.

A broad group of diseases (including respiratory track disorders, infections, cancer, osteoporosis, and metabolic diseases) are treated by either inhalation or intranasal administration of nucleotide or peptide based drugs. The existing technology is designed to delivery drug particles to either the lung or the nasal cavity. Typically, the formulation and nasal spray delivery methods for intranasal drug products are specifically intended to avoid lung deposition, for example, to produce large particles generally greater than 10 microns (see FDA guidance document at http://www.fda.gov/OHRMS/DOCKETS/98fr/99d-1738-gd10002.pdf.pdj). New technology for nasal devices, likewise are intended to avoid lung exposure (see Djupesland, et al, J. Aerosol Med. 17(3):249-59, 2004). Nasal administration of drugs for pulmonary deposition are discussed in Nadithe, et al. and Janssens, et al. (see Nadithe, et al., J. Pharm. Sci. 92(5):1066-76, 2003; Janssens, et al., Chest. 123(6):2083-8, 2003).

As shown by Salmon, et al., nasal inhalation of traditional aerosols may lead to nasal filtration and reduction of dose delivered to the lung (see Salmon, et al., Arch. Dis. Child. 65(4):401-3, 1990). To overcome this effect, Nagai, et al. provides a formulation approach (use of hydroxypropyl cellulose (HPC)) to improve anti-influenza activity of a small molecule (see Nagai, et al., Biol. Pharm. Bull. 20(10):1082-5, 1997).

Current nasal delivery systems include pressurized canisters or Metered-Dose Inhalers (MDI) that eject a drug product into the nostrils in short bursts, or streams of atomized liquid in an aqueous nasal spray. The efficacy of the drug products administered in this manner is limited due to limited diversity in the delivery of drug product. Current systems are limited in particle sizes which prevents combined drug delivery to nasal cavity and pulmonary regions. There is a need to create a droplet size distribution suitable for delivery both to the nasal cavity as well as the pulmonary regions.

### BRIEF DESCRIPTION OF THE DRAWINGS

FIG. 1. A demonstration of a bimodal distribution curve with desirable mean particle sizes that favorably target nasal and pulmonary drug deposition.

FIG. 2. Top view schematics showing several possible nasal spray openings with an asymmetrical, annular orifice to create bimodal and/or broad droplet size distribution.

FIG. 3. A schematic showing that by using two air jets, a unique bimodal droplet size distribution is generated.

FIG. 4. An example of a device for dual-nozzle spray drying to create a powder with a bimodal particle size distribution.

FIG. 5. An example of a device for dual-nozzle and/or dual cyclone spray drying to create a powder with a bimodal particle size distribution.

FIG. 6. An example of a multi-pressure pump actuator controlled by a spring/latch mechanism.

### DETAILED DESCRIPTION

As used herein, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, “about” or “consisting essentially of mean±20% of the indicated range, value, or structure, unless otherwise indicated. As used herein, the terms “include” and “comprise” are used synonymously. It should be understood that the terms “a” and “an” as used herein refer to “one or more” of the enumerated components. The use of the alternative (e.g., “or”) should be understood to mean either one, both or any combination thereof of the alternatives.

In addition, it should be understood that the individual compounds, or groups of compounds, derived from the various combinations of the structures and substituents described herein, are disclosed by the present application to the same extent as if each compound or group of compounds was set forth individually. Thus, selection of particular structures or particular substituents is within the scope of the present disclosure.

The present disclosure fulfills the foregoing needs and satisfies additional objects and advantages by providing a novel, effective method for delivery of a drug product to both the nasal cavity and pulmonary regions.

This disclosure is applicable for treatment of a broad class of diseases including those that are impacted by coverage of the lining of the respiratory tract, such as infections and cancers. The treatments may include infectious, chronic, or congenital diseases.

This disclosure may include administration of a drug through nasal inhalation for the purposes of targeting both the nasal cavity and the respiratory tract including, but not limited to, the upper respiratory tract regions such as naso-, oro-, and laryngo-pharynx; trachea; and bronchial tree.

This disclosure may include delivery of a drug product in a single use format or a multi-use format. A multi-use (e.g., bi-modal, tri-modal, quatra-modal etc.) format (e.g., multi-use bottles) may contain several chambers that connect to different (one or more) spraying mechanisms capable of generating a spectrum of particles of varying sizes, and thus producing a bi- or multi-modal distribution. Such a multi-modal format may also include the ability to deliver formulations of varying compositions, strengths (e.g., in order to titrate a patient dosage).

This disclosure may be used for topical and/or systemic drug delivery, depending on the particle size distribution desired to be achieved. The particle size distribution ranges can be tailored to particular applications for different drugs. A mean particle size greater than about 10 μm is preferred for delivering drugs to the nasal passages. For a pulmonary application, mean particle sizes of less than about 10 μm and particularly between 5-10 μm are preferred. Particles below about 3 μm in size can be generated for deep lung and systemic drug delivery.

Some peptides and proteins can be administered intranasally using a nasal spray or aerosol. This is surprising because many proteins and peptides have been shown to be sheared or denatured due to the mechanical forces generated by the actuator in producing the spray or aerosol. This disclosure includes a method to administer an appropriately formulated drug product through a delivery device to both the nasal cavity and pulmonary regions. This disclosure may be used to deliver small molecule drugs and biologics, including nucleotide or peptide based drugs. An example includes the biomodal delivery of therapeutic siRNA to the nasal and pulmonary regions of an influenza infected patient.

This disclosure allows combined topical and systemic drug delivery via the nasal and pulmonary routes for a wide variety of drugs that can be formulated or prepared in situ or immediately before use as solution, suspension or emulsion or any other pharmaceutical application system. Multiple droplet and/or particle sizes can be generated to achieve bimodal delivery.

Various drugs can be administered as formulations with immediate or controlled drug release. Alternatively, the drug can be formulated as a vesicle such as a liposome or nanosome, or as a micro and/or nanocapsule. This disclosure is useful for the application of most all therapeutic drug classes alone or in combinations. Drugs can be formulated as any pharmaceutical acceptable derivative or salt.

An alternative to the formulation approach (HPC) for improving anti-influenza activity of a small molecule is to intentionally create a droplet size distribution suitable for delivery both to the nasal cavity as well as the pulmonary regions. For purposes of targeting the upper respiratory tract, particle administration includes a broad (Gaussian) distribution covering particle sizes from 2 to 100 μm. Alternatively, the particles may be bimodal in distribution with, for example, a mean particle diameter in the 5-10 μm range and a mean particle diameter in the 30-60 μm range. Such bimodal particle size distribution may include bimodal particles in a range from about 1 μm to about 10 μm and particles in a range from about 10 μm to about 80 μm; particles in a range from about 5 μm to about 10 μm and particles in a range from about 10 μm to about 80 μm; particles in a range from about 2 μm to about 6 μm and particles in a range from about 30 μm to about 60 μm. Very broad particle size distribution ranges are also included.

A range is typically described as “span” which is further defined as (Dv,90-Dv,10)/Dv,50 where Dv,10 Dv,50 and Dv,90 are the diameters at 10%, 50% and 90% of the particle volume distribution. For example, consider a bimodal distribution, one mode with Dv,50 at 3-5 microns and the other with Dv,50 at 30-60 microns. In order to avoid substantial overlap of the two distributions, the span should preferably be less than 5, for example in the range of 1 to 5. Ideally the span would be in the range of 1 to 3, for example, a span of 2. In some embodiments herein, a single wide Gaussian distribution curve to cover both nasal and pulmonary droplet sizes, the span may be large, for example greater than 5, including 6, 7, 8, 9, 10 and so on. As an illustration, if both distributions had a span of 3, possible values for the first and second distributions could have Dv10 Dv50 and Dv,90 of 1, 3, and 10 and 10, 40, and 130. As a further illustration if both distributions had a span of 2, possible values for the first and second distributions could have Dv,10 Dv,50 and Dv,90 of 1, 4, and 9 and 10, 40, and 90. As an illustration if both distributions had a span of 1, possible values for the first and second distributions could have Dv,10 Dv,50 and Dv,90 of 1, 2, and 3 and 20, 40, and 60.

The particles may be comprised of a nebulized solution or powder and are intended to lodge along the entire upper and possibly lower or deep respiratory tract. The dry powders may be generated by various processes such as spray drying with dual nozzles, spray freeze drying with dual nozzles or create a partially friable spray freeze dried powder with a dual particle size distribution, or by blending of milled freeze-dried or milled powders of two different particle sizes.

The particles may be generated in situ via a device or an actuator consisting of dual nozzles for the bimodal distribution, or a specially designed nozzle to generate a broad particle size distribution for the Gaussian size range. Approaches to generate bimodal and/or broad Gaussian distribution include asymmetric nozzle orifice or time-delayed actuation across the nozzle.

The following definitions are useful:

1. Aerosol—A product that is packaged under pressure and contains therapeutically active ingredients that are released upon activation of an appropriate valve system.

2. Metered aerosol—A pressurized dosage form comprised of metered dose valves, which allow for the delivery of a uniform quantity of spray upon each activation.

3. Powder aerosol—A product that is packaged under pressure and contains therapeutically active ingredients in the form of a powder, which are released upon activation of an appropriate valve system.

4. Spray aerosol—An aerosol product that utilizes a compressed gas as the propellant to provide the force necessary to expel the product as a wet spray; it generally applicable to solutions of medicinal agents in aqueous solvents.

5. Spray—A liquid minutely divided as by a jet of air or steam. Nasal spray drug products contain therapeutically active ingredients dissolved or suspended in solutions or mixtures of excipients in nonpressurized dispensers.

6. Metered spray—A non-pressurized dosage form consisting of valves that allow the dispensing of a specified quantity of spray upon each activation.

7. Suspension spray—A liquid preparation containing solid particles dispersed in a liquid vehicle and in the form of course droplets or as finely divided solids.

The fluid dynamic characterization of the aerosol spray emitted by metered nasal spray pumps as a drug delivery device (“DDD”). Thorough characterization of the spray's geometry is an indicator of the overall performance of nasal spray pumps. In particular, measurements of the spray's divergence angle (plume geometry) as it exits the device; the spray's cross-sectional ellipticity, uniformity and particle/droplet distribution (spray pattern); and the time evolution of the developing spray have been found to be the most representative performance quantities in the characterization of a nasal spray pump. During quality assurance and stability testing, plume geometry, pump delivery, droplet size, and spray pattern measurements are key identifiers for verifying consistency and conformity with the approved data criteria for the nasal spray pumps.

The following definitions are useful:

Plume Height—the measurement from the actuator tip to the point at which the plume angle becomes non-linear because of the breakdown of linear flow. Based on a visual examination of digital images, and to establish a measurement point for width that is consistent with the farthest measurement point of spray pattern, a height of 30 mm is defined for this study.

Major Axis—the largest chord that can be drawn within the fitted spray pattern that crosses the COMw in base units (mm).

Minor Axis—the smallest chord that can be drawn within the fitted spray pattern that crosses the COMw in base units (mm).

Ellipticity Ratio—the ratio of the major axis to the minor axis, preferably between 1.0 and 1.5, and most preferably between 1.0 and 1.3.

D10—the diameter of droplet for which 10% of the total liquid volume of sample consists of droplets of a smaller diameter (μm).

D50—the diameter of droplet for which 50% of the total liquid volume of sample consists of droplets of a smaller diameter (μm), also known as the mass median diameter.

D90—the diameter of droplet for which 90% of the total liquid volume of sample consists of droplets of a smaller diameter (μm).

Span—measurement of the width of the distribution, the smaller the value, the narrower the distribution. Span is calculated as:

(D90−D10)/D50

% RSD—percent relative standard deviation, the standard deviation divided by the mean of the series and multiplied by 100, also known as % CV.

Volume—the volume of liquid or powder discharged from the delivery device with each actuation, preferably between 0.01 mL and about 2.5 mL and most preferably between 0.02 mL and 0.25 mL.

The following examples are provided by way of illustration, not limitation.

### EXAMPLE 1

### Methods of Creating Bimodal and/or Broad Droplet Size Distribution That Targets Both Nose and Upper Respiratory Tract for Drug Delivery

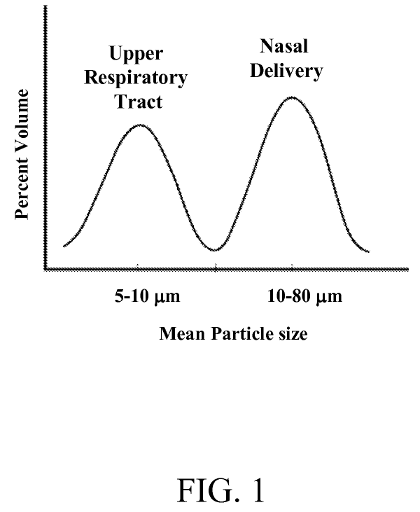

FIG. 1 illustrates a bimodal aerodynamic particle size distribution curve that can be generated by the methods described below. The bimodal distribution described in FIG. 1 includes mean particle size within the 10-80 μm range for intranasal delivery and mean particle size within the 5-10 μm range for upper respiratory track delivery.

### Method 1: An Asymmetric Orfice Actuator Opening

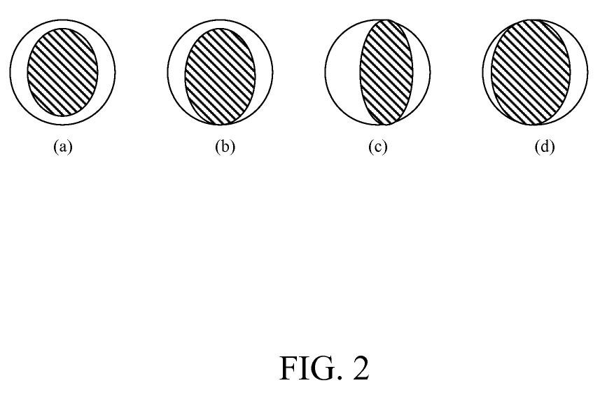

One method of creating bimodal and/or broad droplet size distribution that targets both nose and upper respiratory tract for drug delivery is a design for an asymmetric orifice actuator opening that produces bimodal and broad droplet size distribution. Four asymmetric opening designs for a nasal delivery device with an asymmetrical annular orifice to produce a bimodal and/or broad droplet size distribution are shown in FIG. 2. FIG. 2 shows model actuator openings from the top view. The diagonal-lined area indicates a cross section of a round solid component whereas the open (no diagonal lines) areas are the annular orifice openings. The first two scenarios (a and b) are designed to give a broad range distribution whereas the other two scenarios (c and d) give a more distinct, bimodal peak distribution (similar to FIG. 1). The annular orifice opening can be in the range of 0.01 to 1 mm, or preferably from 0.05 to 0.5 mm.

### Method 2: High-velocity Air Jets to Atomize a Liquid Formulation

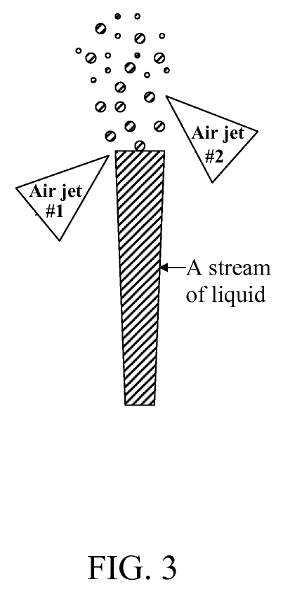

Another method of creating bimodal and/or broad droplet size distribution that targets both the nose and upper respiratory tract for drug delivery is a design for a device that uses a high-velocity air jet to atomize the liquid formulation to produce bimodal droplet size distribution. FIG. 3 shows the use of a high-velocity air jet to atomize liquid to form liquid droplets with a unique bimodal droplet size distribution for nasal and lung delivery. The air source is preferably sterile, passing through an approximately 0.2 μm filter before atomizing with the formulation liquid. The optimum velocities to generate the liquid droplet sizes that are desirable for both nasal and pulmonary delivery for maximum coverage are determined experimentally for each formulation. A range for generating smaller particle sizes that target both nasal and lung deposition includes a velocity from 10-70 mm/s for nasal deposition and a range of 70-200 mm/s for lung deposition. FIG. 3 shows two air jets atomizing a liquid stream. Air jet #1 first breaks up the liquid into larger particle sizes (approximately 10-80 μm range) whereas the air jet #2 further breaks down the droplets into smaller particle sizes (approximately 5-10 μm range). Some of the larger particles created by air jet #1 escape air jet #2 and thus a mix of various particles sizes with bimodal peak distribution is generated. The air velocities of air jet #1 and #2 can be the same or different.

The volume amounts of the different particle sizes in the mix do not need to be equal, one can engineer the orifice sizes or the air jets (either by speed or position) to produce different volume percentage mixes. For example, multiple droplet size distributions can be created by using polygon (instead of round) solid components in the middle of an orifice or by using multiple (instead of two) air jets.

### Method 3: Nanoparticle Size Distribution in Formulation Suspension

Another method of creating bimodal particles is via nanoparticle size distribution in formulation suspension. Generally, particles are formed via process and formulation size controls. Process size controls may include physical or mechanical based processes such as jet milling or ball milling, and formulation controls may include chemical approaches such as changing excipients or the order of ingredients. The particle size control can be performed via several operations such as jet milling or ball milling for solid crystalline particle Active Pharmaceutical Ingredients (APIs) as is commonly performed for small molecule drugs.

Particles are incorporated in a dual or broad Gaussian distribution to allow a high energy or pressure atomization for complete dispersion of the particles. The particles for delivery to the upper deep lung will be of a size range from about 1-10 μm and the particles for nasal delivery will be particles about >˜10 μm and <˜80 μm.

Alternatively, different processes can be used, such as poly(lactic-co-glycolic acid) (PLGA) microspheres to make dual distribution particles. For other nanoparticles, it may be desirable to formulate sizes within the desired range using various amounts of material or processes to arrive at the target size range. The different sized particles are then combined to form a single suspension for dosing.

Also described is the preparation of separate batches of particles with different particle size distributions followed by their mixing. Two or more batches of particles may be combined to create the bimodal particle size distribution. The two or more batches of dried particles can be produced by a variety of techniques including, but not limited to spray drying to create dense primary particles (see Masters, K., Spray Drying Handbook, John Wiley and Sons, New York, N.Y., 1991); spray drying to create low-density particles (see Edwards, D. A., et al., “Aerodynamically Light Particles for Pulmonary Drug Delivery,” U.S. Pat. No. 6,977,087); freeze drying (see H. R. Costantino, et al., “Lyophilization of Biomaterials,” AAPS Press, Washington, D.C., eds. 2004); followed by a milling technique (see Tracy, M. A., et al., U.S. Pat. No. 6,713,087); spray freeze drying (see Costantino, H. R., et al., U.S. Pat. No. 6,428,815); fluid bed drying (see Yang W-C, and Y. Yang, Handbook of Fluidization and Fluid-Particle Systems, Marcel Dekker, New York); spray freezing into liquid (Williams, R. O., et al., U.S. patent application Ser. No. 10/273,730); and evaporation precipitation (see Johnson, K. P., et al., U.S. patent application Ser. No. 10/266,998). One or more of the batches to be mixed is comprised of particles with a mass median aerodynamic diameter in the range of 1-10 microns, more preferably in the range of 2-6 microns and the batch(es) are mixed with one or more additional batch(es) of particles with a mass median aerodynamic diameter in the range of 10-100 microns, more preferably in the range of 30-60 microns. The resulting mixture can be accomplished by a variety of mixing techniques known in the art (see Kaye, B. H., “Powder Mixing,” Chapman & Hall, London, 1997). The mixture of particles can be blended and coated onto larger carrier particles, for example lactose particles with a mass median diameter in the range of 100-300 microns (see Adjei, A. L. and P. K. Gupta, “Inhalation Delivery of Therapeutic Peptides and Proteins,” Marcel Dekker (eds.), New York, 1997).

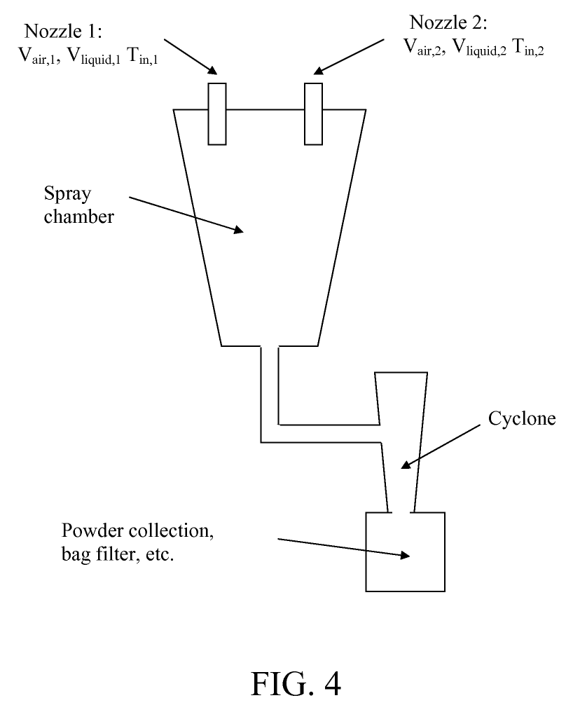

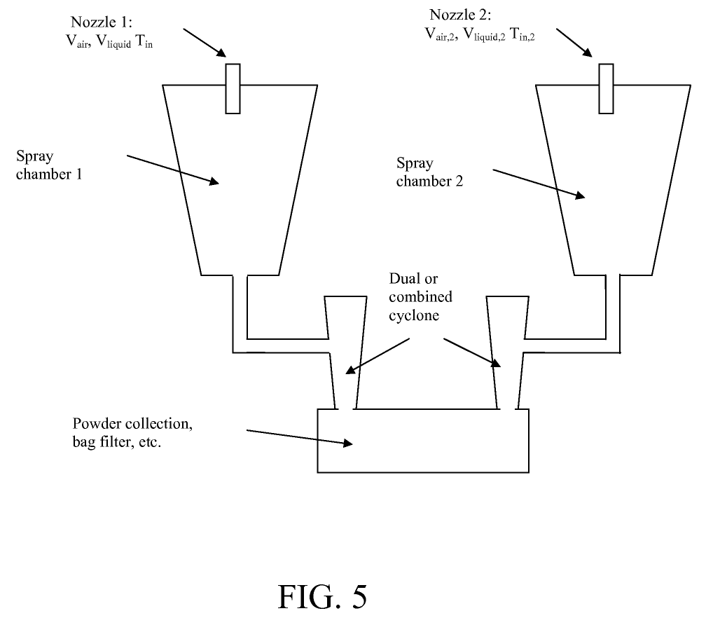

Also described is a bi- or multimodal dry particle size distribution that is created as a single batch, as opposed to create of separate batches of different sized particles followed by mixing. The creation of the bimodal dry particle size distribution can be achieved by different techniques. One example is by spray freeze drying followed by fragmentation of the friable particles (see Costantino, H. R., et al., U.S. Pat. No. 6,428,815). Another example would be a dual-nozzle spray drying process, as shown, for example in FIG. 4 where a single drying chamber is used, or for example in FIG. 5 where dual drying chambers and/or dual cyclones are used. In either case shown by FIG. 4 and FIG. 5, the nozzle conditions for nozzle #1, specifically the air inlet flow rate (V_{air,1}) and liquid inlet flow rate (V_{liquid,1}) and the air inlet temperature (T_{in,1}) are optimized to generate particles with a mass median aerodynamic diameter in the range of 1-10 microns, more preferably in the range of 2-6 microns and the nozzle conditions for nozzle #2, specifically the the air inlet flow rate (V_{air,2}) and liquid inlet flow rate (V_{liquid,2}) and the air inlet temperature (T_{in,2}) are optimized to generate particles with a mass median aerodynamic diameter in the range of 10-100 microns, more preferably in the range of 30-60 microns.

### Method 4: Multi-pressure Pump Actuator with Spring/Latch Mechanism

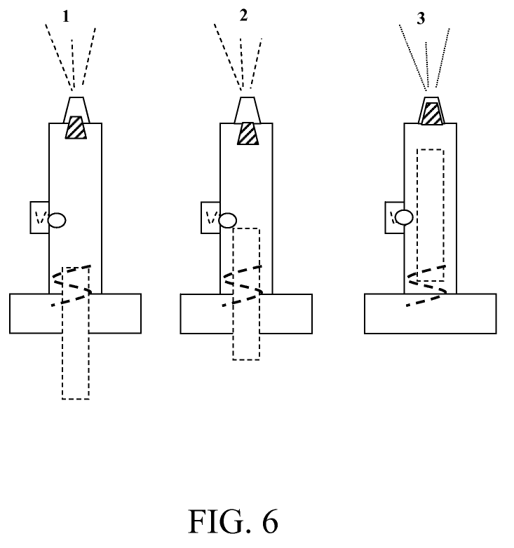

The bimodal particles described in FIG. 1 can be created via a multi-pressure pump actuator controlled by a spring/latch mechanism such as the actuator depicted in FIG. 6. Using the spring/latch mechanism, one can use a spring activated actuator which has two specific pressures that are created by a pressure regulated pin that achieves a high pressure spray though a nozzle with an orifice of fixed size or of varying size as described above.

The various stages for bimodal particle delivery using the device shown in FIG. 6 are described as: (1) Low pressure “standard” actuation resulting in larger droplet sizes. The droplet sizes would be in the range of 10 μm to 80 μm intended primarily for nasal coverage. (2) The spring actuated ball would allow a pressure buildup during the pumping of the actuator. This would permit a large degree of force to be built up in the lower spring which is released once the ball is pushed into the spring chamber. (3) Once the piston overcomes the force it ejects upward with a significantly higher degree of force. The high pressure that was built up during actuation results in the forceful ejection of the solution through the nozzle. At the same time, the internal nozzle regulator (diagonal-lined in FIG. 6) is spring activated to pinch the orifice and create a small opening that allows shearing of the particles resulting in the small particle size droplets, 3-10 μm in diameter necessary for lung deposition.

Although the foregoing disclosure has been described in detail by way of example for purposes of clarity of understanding, it will be apparent to the artisan that certain changes and modifications are comprehended by the disclosure and may be practiced without undue experimentation within the scope of the appended claims, which are presented by way of illustration not limitation.

All U.S. patents, U.S. patent application publications, U.S. patent applications, foreign patents, foreign patent applications, non-patent publications, figures, tables, and websites referred to in this specification are expressly incorporated herein by reference, in their entirety.

What is claimed is:

1. A device for delivery of a pharmaceutical formulation, comprising a nasal actuator with a asymmetric orifice opening that produces bimodal particle size distribution.

2. The device of claim 1, wherein the bimodal particle size distribution ranges include 1-10 μm and 10-100 μm.

3. The device of claim 1, wherein the bimodal particle size distribution ranges include 5-10 μm and 10-80 μm.

4. The device of claim 1, wherein the bimodal particle size distribution ranges include 2-6 μm and 30-60 μm.

5. The device of claim 1, wherein the asymmetric orifice opening is in the range of approximately 0.01 to 1 mm.

6. The device of claim 1, wherein the asymmetric orifice opening is in the range of approximately 0.05 to 0.5 mm.

7. A method for delivering a pharmaceutical formulation to both nasal cavity and pulmonary regions, comprising a nasal actuator with an asymmetric orifice opening that produces bimodal particle size distribution.

8. The method of claim 7, wherein the bimodal particle size distribution ranges include 1-10 μm and 10-100 μm.

9. The method of claim 7, wherein the bimodal particle size distribution ranges include 5-10 μm and 10-80 μm.

10. The method of claim 7, wherein the bimodal particle size distribution ranges include 2-6 μm and 30-60 μm.

11. The method of claim 7, wherein the asymmetric orifice opening is in the range of approximately 0.01 to 1 mm.

12. The method of claim 7, wherein the asymmetric orifice opening is in the range of approximately 0.05 to 0.5 mm.

13. A device for delivery of a liquid pharmaceutical formulation, comprising a nasal actuator and one or more high-velocity air jets to atomize the liquid formulation to produce bimodal droplet size distribution.

14. The device of claim 13, wherein the bimodal droplet size distribution ranges include 1-10 μm and 10-100 μm.

15. The device of claim 13, wherein the bimodal droplet size distribution ranges include 5-10 μm and 10-80 μm.

16. The device of claim 13, wherein the bimodal droplet size distribution ranges include 2-6 μm and 30-60 μm.

17. A method for delivering a pharmaceutical formulation to both nasal cavity and pulmonary regions, comprising a nasal actuator and one or more high-velocity air jets to atomize the liquid formulation to produce bimodal droplet size distribution.

18. The method of claim 17, wherein the bimodal droplet size distribution ranges include 1-10 μm and 10-100 μm.

19. The method of claim 17, wherein the bimodal droplet size distribution ranges include 5-10 μm and 10-80 μm.

20. The method of claim 17, wherein the bimodal droplet size distribution ranges include 2-6 μm and 30-60 μm.

21. A method for delivery of a pharmaceutical formulation, comprising nanoparticle size distribution in formulation suspension for creating bimodal particle size distribution.

22. The method of claim 21, wherein the bimodal particle size distribution ranges include 1-10 μm and 10-100 μm.

23. The method of claim 21, wherein the bimodal particle size distribution ranges include 5-10 μm and 10-80 μm.

24. The method of claim 21, wherein the bimodal particle size distribution ranges include 2-6 μm and 30-60 μm.

25. A device for delivery of a pharmaceutical formulation, comprising a multi-pressure pump nasal actuator with a spring/latch mechanism that produces bimodal particle size distribution.

26. The device of claim 25, wherein the bimodal particle size distribution ranges include 1-10 μm and 10-100 μm.

27. The device of claim 25, wherein the bimodal particle size distribution ranges include 5-10 μm and 10-80 μm.

28. The device of claim 25, wherein the bimodal particle size distribution ranges include 2-6 μm and 30-60 μm.

29. A method for delivering a pharmaceutical formulation to both nasal cavity and pulmonary regions, comprising a multi-pressure pump nasal actuator with a spring/latch mechanism that produces bimodal particle size distribution.

30. The method of claim 29, wherein the bimodal droplet size distribution ranges include 1-10 μm and 10-100 μm.

31. The method of claim 29, wherein the bimodal droplet size distribution ranges include 5-10 μm and 10-80 μm.

32. The method of claim 29, wherein the bimodal droplet size distribution ranges include 2-6 μm and 30-60 μm.

33. A means for creating bimodal particle size distribution that targets both nasal cavity and pulmonary regions for drug delivery.

34. A means for delivering particles with peak particle size distribution in the ranges of 1-10 μm and 10-100 μm.

35. A means for delivering particles with peak particle size distribution in the ranges of 5-10 μm and 10-80 μm.

36. A means for delivering particles with peak particle size distribution in the ranges of 2-6 μm and 30-60 μm.
